(19) United States
(12) Patent Application Publication
(10) Pub. No.: US 2009/0306779 A1
(43) Pub. Date: Dec. 10, 2009

(54) Modular anterior locking interbody cage

(75) Inventor: Uri AHN, Bedford, NH (US)

Correspondence Address: ALPHATEC SPINE, INC., 5818 EL CAMINO REAL, CARLSBAD, CA, 92008, US

(73) Assignee: ALPHATEC SPINE, INC., Carlsbad, CA (US)

(21) Appl. No.: 12/455,719

(22) Filed: Jun. 5, 2009

Related U.S. Application Data
(60) Provisional application No. 61/059,181, filed on Jun. 5, 2008.

(51) Int. Cl. A61F 2/44

(52) U.S. Cl. 623/17.11

(57) ABSTRACT

Apparatus and method if using a modular spinal implant system for use between adjacent vertebrae near vascular anatomy. The system includes an implant configured the fit between adjacent vertebrae, the implant having annular side walls with upper and lower surfaces configured to enclose a hollow interior, and an attachment plate rotatably coupled to the implant and configured to rotate to variable orientations relative to the implant to avoid the vascular anatomy, the attachment plate having a superior portion that is narrower than an inferior portion, the attachment plate having at least one vertebra attachment hole configured for attaching to at least one adjacent vertebrae using one or more bone screws.

### CROSS-REFERENCES TO RELATED APPLICATIONS

The present application claims priority to U.S. Provisional Patent Application No. 61/059,181 to Ahn, filed Jun. 5, 2008, and entitled “MODULAR ANTERIOR LOCKING INTERBODY CAGE”, the disclosure of which is incorporated herein by reference in its entirety.

### BACKGROUND OF THE INVENTION

1. Field of the Invention

The present invention is directed to systems, methods, and devices applicable to spinal surgery. More specifically, the present invention is directed to a modular spinal spacer designed to accommodate the vascular anatomy for use by medical personnel (i.e., doctor) in spinal and other surgical procedures. In some embodiments of the present invention relates to a modular spinal spacer for insertion into a disk space defined between two adjacent vertebrae near vascular anatomy, in order to restore an appropriate height between the vertebrae and to allow bone fusion to take place between adjacent vertebrae.

2. Background of the Invention

Vertebrae are the individual irregular bones that make up the spinal column (aka ischis)—a flexuous and flexible column. There are normally thirty-three vertebrae in humans, including the five that are fused to form the sacrum (the others are separated by intervertebral discs) and the four coccygeal bones which form the tailbone. The upper three regions comprise the remaining 24, and are grouped under the names cervical (7 vertebrae), thoracic (12 vertebrae) and lumbar (5 vertebrae), according to the regions they occupy. This number is sometimes increased by an additional vertebra in one region, or it may be diminished in one region, the deficiency often being supplied by an additional vertebra in another. The number of cervical vertebrae is, however, very rarely increased or diminished.

A typical vertebra consists of two essential parts: an anterior (front) segment, which is the vertebral body; and a posterior part—the vertebral (neural) arch—which encloses the vertebral foramen. The vertebral arch is formed by a pair of pedicles and a pair of laminae, and supports seven processes, four articular, two transverse, and one spinous, the latter also being known as the neural spine.

When the vertebrae are articulated with each other, the bodies form a strong pillar for the support of the head and trunk, and the vertebral foramina constitute a canal for the protection of the medulla spinalis (spinal cord), while between every pair of vertebrae are two apertures, the intervertebral foramina, one on either side, for the transmission of the spinal nerves and vessels.

Conventional interbody spacer assemblies are used in spinal fusion procedures to repair damaged or incorrectly articulating vertebrae. Conventional interbody spacer assemblies come in different cross sections. Some spacer assemblies may be hollow and may include openings in the side(s) thereof to provide access for bone matter growth. The use of interbody spacers are primarily to support the anterior load of the spinal column and provide a method for insertion and containment of bone graft material to facilitate spinal fusion. Often these spacers are used in conjunction with supplemental fixation in the form of pedicle screws or anterior plate systems.

Historically one of the failure modes of interbody spacers used in combination with anterior plate systems particularly in the lumbar spine in one of placing the anterior plate due to the vascular system lying directly over the area of interest. This has been previously addressed by surgically mobilizing the vascular structures or particularly in the upper lumbar levels avoiding the use of anterior lumbar plate's altogether and utilizing posterior supplemental instrumentation. Some implant designs have integrated features that provide for integrated supplemental fixation such as spikes, protrusions, screws, once installed but generally do not provide the same level of rigidity as a plate creating a paradoxical relationship where implant manufacturers must choose between either making the implant system easier to insert or making the implant system more effective in stabilize the spine to facilitate fusion.

There exists a need for further improvements in the field of spinal spacer assemblies of the present type that are designed to avoid the vascular structures.

### BRIEF SUMMARY OF THE INVENTION

In a first aspect, embodiments of the present invention provide a modular spinal implant system for use between adjacent vertebrae near vascular anatomy. The system includes an implant configured to fit between adjacent vertebrae, the implant having annular side walls with upper and lower surfaces configured to enclose a hollow interior, and an attachment plate rotatably coupled to the implant and configured to rotate to variable orientations relative to the implant to avoid the vascular anatomy, the attachment plate having a superior portion that is narrower than an inferior portion, the attachment plate having at least one vertebra attachment hole configured for attaching to at least one adjacent vertebrae using one or more bone screws.

In many embodiments, the attachment plate is selected from a variety of attachment plates configured to avoid the vascular anatomy proximate the vertebrae.

In many embodiments, the inferior portion includes one or more vertebra attachment holes.

In many embodiments, the superior portion includes one vertebra attachment hole and the inferior portion includes two vertebra attachment holes.

In many embodiments, the implant material is selected from the group consisting of titanium, stainless steel, cobalt-chromium, carbon, PEEK (polyethylketone), graphite, woven carbon, Kevlar, and other suitable synthetic material.

In many embodiments, the implant is made of a non metal synthetic material. In further embodiments, the implant further includes one or more metal plates integrally formed within an anterior portion of the annular side wall

In many embodiments, the attachment plate is made from titanium, stainless steel, or cobalt-chromium.

In many embodiments, mating surfaces between the attachment plate and implant include an interlock configuration.

In many embodiments, the system further includes a bone autograft, allograft or a bone graft substitute positioned within the hollow interior of the implant.

In another aspect, embodiments of the present invention provide a method of installing a modular spinal implant system between adjacent vertebrae. The method includes inserting the modular spinal implant system between adjacent vertebrae, the system includes an implant configured to fit between adjacent vertebrae, the implant having annular side walls with upper and lower surfaces configured to enclose a hollow interior and an attachment plate rotatably coupled to the implant before, during, or after implantation and configured to rotate to variable orientations relative to the implant to avoid the vascular anatomy, the attachment plate having a superior portion that is narrower than an inferior portion, the attachment plate having at least one vertebra attachment hole configured for attaching to at least one adjacent vertebrae using one or more bone screws, rotating the attachment plate to avoid the vascular anatomy, and attaching the attachment plate to at least one adjacent vertebra using one or more bone screws.

In many embodiments, the attachment plate is selected from a variety of attachment plates configured to avoid the vascular anatomy proximate the vertebrae.

In many embodiments, prior to inserting the system, the method further includes retracting a portion of the vascular anatomy.

In many embodiments, the implant material is selected from the group consisting of titanium, stainless steel, cobalt-chromium, carbon, PEEK (polyethylketone), graphite, woven carbon, Kevlar, and other suitable synthetic material.

In many embodiments, the method further includes filling the hollow interior with bone autograft, allograft or a bone graft substitute.

### BRIEF DESCRIPTION OF THE DRAWINGS

FIG. 1 shows a top view of one embodiment of an oval shaped body or cage.

FIG. 2 shows a top view of a “D” shaped body.

FIG. 3A shows a L5/S1 anterior view of a modular anterior locking interbody cage (MALIC) system in place between vertebrae proximate vascular structures.

FIG. 3B shows one embodiment of attaching a plate to the implant.

FIG. 4A shows one example of an anterior view of the spine in which a vascular portion may require retraction for implantation and attachment of an attachment plate.

FIG. 4B shows retraction of the vascular portion.

FIG. 4C-4E show different sizes and shapes of attachment plates to attach to an implant that may be used to avoid the vascular.

FIG. 5 shows examples of surface treatment of the plate and implant where they join.

FIG. 6A shows a top view of one embodiment an implant that is a non metal synthetic material (NMSM)/metal amalgam.

FIGS. 6B-6E show other embodiments of a NMSM/metal amalgam implant.

FIG. 7 shows a view of a lateral x-ray showing an implant of FIG. 6A positioned within between adjacent vertebrae.

### DETAILED DESCRIPTION OF THE INVENTION

One or more detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

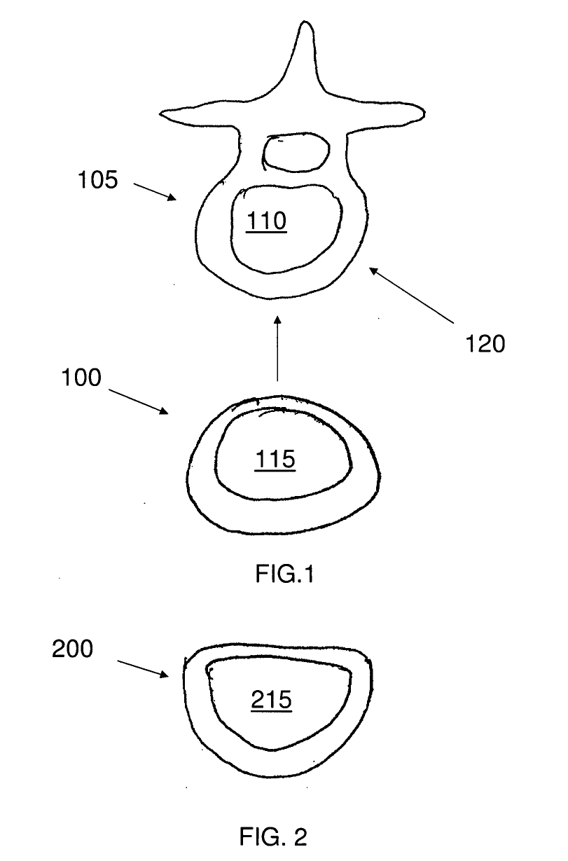

FIG. 1 shows one embodiment of an anterior locking interbody cage for use as an anterior interbody fusion device 100 in the lumbar spine 105. The disc interspace is shaped like a “D” 110. The device 100 has a generally oval or circular shaped body or cage, when viewed from above, having an annular wall enclosing a hollow interior or area 115 that would permit bony growth into spinal bones above and below the device when implanted. The hollow area 115 would be filled with bone autograft, allograft or a bone graft substitute.

FIG. 2 shows a “D” shaped body 200 body or cage, when viewed from above, having an annular wall enclosing a hollow interior or area 215 that would permit bony growth into spinal bones above and below the device when implanted. The hollow area 215 would be filled with bone autograft, allograft or a bone graft substitute.

There are some advantages using the oval or circular shape shown in FIG. 1 over “D” shape 200 shown in FIG. 2. For example, the “D” shaped device can only be placed in the disc space in one orientation. The “D” shape has posterior corners that can impinge on the aortic/venous iliac vessels. The “D” design is not suitable for a lateral approach 120. In contrast, an oval shaped device permits more implant options, so that the device could be placed in different rotational orientations within the spine, and would have the advantage of sliding safely past aortic/venous iliac vessels. The oval shaped device 100 also allows variability to approach the disc space from multiple angles 120, again permitting variability to better accommodate the vascular anatomy, such as. For example, oval shaped device may also be placed into patients through a lateral approach 120, which a “D” design does not afford, the lateral approach being favored by many surgeons in an anterior approach to L4/5.

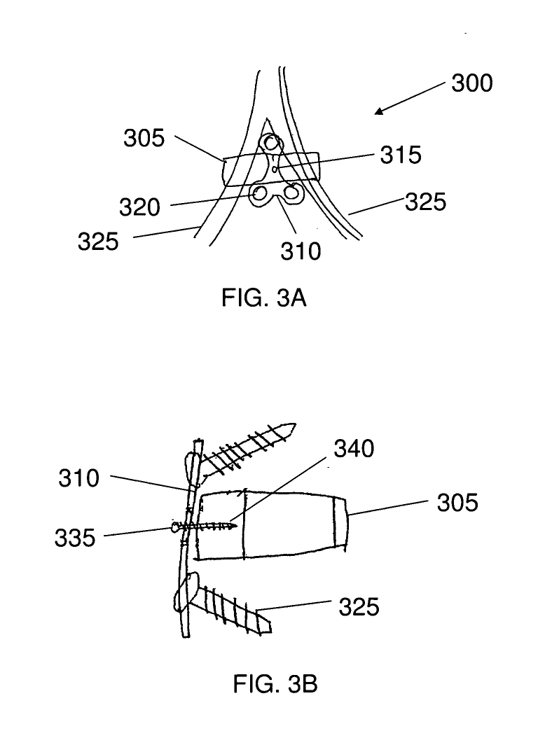

FIG. 3A shows a L5/S1 anterior view of one embodiment of a modular anterior locking interbody cage (MALIC) system 300 configured to be placed between vertebra. The system 300 includes an interbody fusion device or implant 305 and an attachment plate 310. The implant 305 may be made of a metal, a non metal synthetic material, or a NMSM/metal amalgam, discussed below. The attachment plates 310 are coupled to the implant with a screw, and would allow a variation of attachment plates 310 to be attached onto the implant 305, making the system 300 modular. The attachment plates are designed to accommodate the vascular 325 anatomy. Using a screw or other fixation means such as a rivet or snap locking mechanism for attachment with the implant allows the attachment plate to rotate to various orientations to avoid the vascular anatomy. The attachment plate may be coupled to the implant either before, during, or after implantation. The screw attachment also allows the attachment plate to be removable from the body in case it needs to be replaced or repositioned.

The attachment plate 310 has a superior portion (anatomically cranial) and an inferior portion (anatomically caudal). The superior portion is narrower than the inferior portion. The attachment plate 310 in turn would have holes that would allow the placement of bone screws 320 into the vertebra above and/or below, locking the implant 305 in place. The attachment plate 310 may be made from metal, such as titanium, stainless steel or cobalt-chromium. The attachment plate 310 may also be made from high strength composites or plastics such as PEEK.

In addition, the attachment plate 310 adjacent to the device 305 may have a contouring that would allow a male/female counterpart contouring on a front surface of the device. This would allow a surface interlock that would resist rotational forces between the attachment plate 310 and the device 305. The primary reason for the attachment plate 310 shape is the complexity of the vascular anatomy, especially at the spinal levels superior to L5-S1, that can make access in one area of the spine easier than another. This would allow the surgeon a variety of attachment plates 310 to choose from, selecting the best shape to accommodate the complex vascular anatomy. By creating this modularity in attachable plates this device would have a variety of attachable plates that would accommodate different vascular anatomic challenges, allowing surgery to be performed in a safer manner. This would be done without sacrificing biomechanical strength and the plate would in turn lock onto the MALIC.

FIG. 3B is a side view showing one embodiment of attaching the attachment plate 310 to the implant 305. Screw 335 attaches the attachment plate 310 to the implant 305, in particular, attaching to the embedded metal plate 340 within the implant 305. Bone screws 325 are then used to attach the attachment plate to the vertebra above and/or below implant 305.

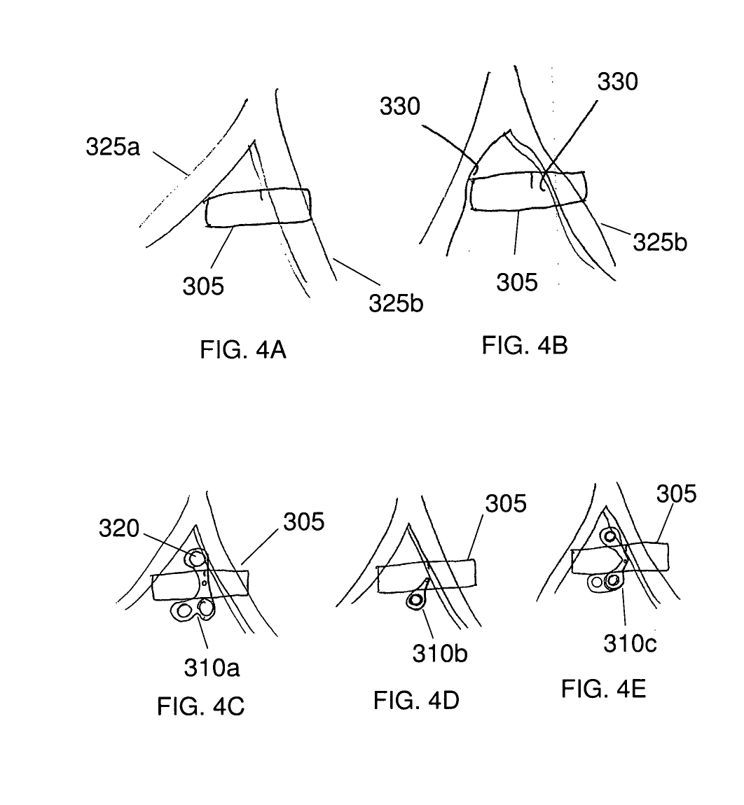

FIG. 4A shows one example of an anterior view of the spine in which a vascular portion requires retraction for implantation and attachment of an attachment plate 310 to a body 305 and adjacent vertebrae. In this example of the vertebral levels above L5-S1 area, the vascular portion 325 is draped over the left side of the vertebrae. The vascular 325a and/or 325b is retracted 330, such as shown in FIG. 4B, to make room for the attachment plate 310 to attach to the implant 305 and vertebrae. FIG. 4C-4E show different sizes and shapes of attachment plates 310 that may be used to avoid the vascular, having superior portions narrower than inferior portions. In FIG. 4C, attachment plate 310a may include provisions for one screw attaching to a vertebra above the implant 305 and two screws attaching to a vertebra below the implant 305. In FIG. 4D, attachment plate 310b attaches to a vertebra below the implant 305. In FIG. 4E, attachment plate 310c may include provisions for one screw attaching a vertebra above the implant 305 and two screws attaching to a vertebra below the implant 305.

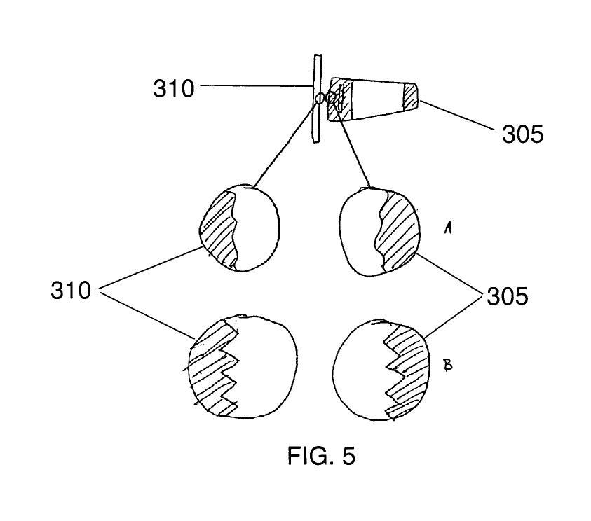

In some cases, it may be desirable to have surface treatment of the attachment plate 310 and implant 305 where they join. For example, FIG. 5 shows two examples A and B. The mating surfaces in A have adjacent irregular contouring and B have regular pyramidal male/female interlocking features to provide additional stability of the assemble components that may include rotational stability.

### Non Metal Synthetic Material/Metal Amalgam

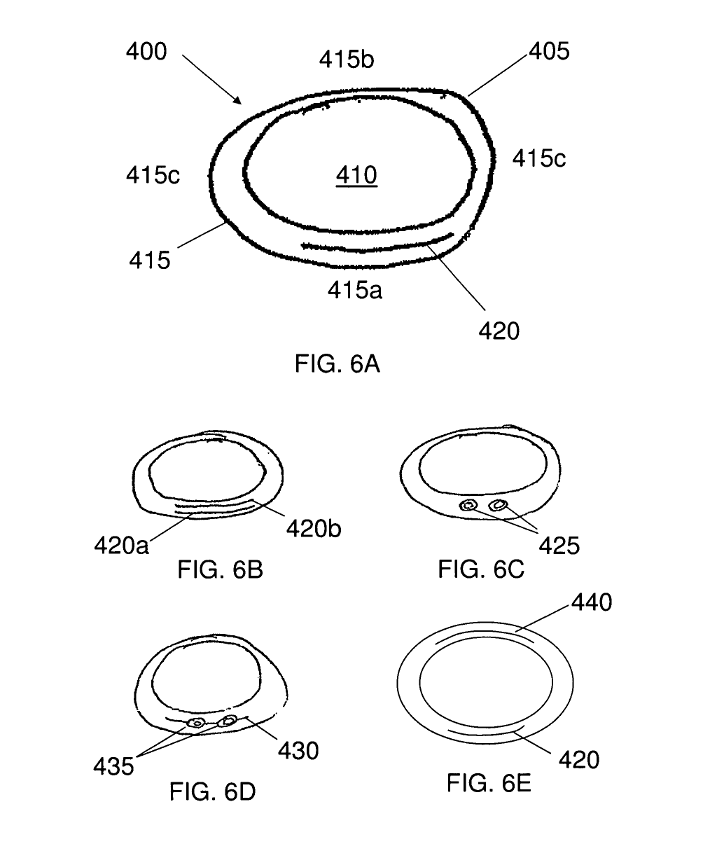

FIG. 6A shows one embodiment an implant 400, having a generally oval or circular shape similar to device 100, with an amalgam body of non metal synthetic material (NMSM) and metal material. In some embodiments, implant 400 may be used in place of implant 300 in the systems describe above. The device 400 includes an oval body 405 or cage with an annular wall 415 having upper and lower surfaces enclosing a central opening 410 or hollow interior. The upper and lower surfaces are configured to contact adjacent spine member and may have raised ridges projecting outwardly from each of the surfaces for engaging the spinal column. The annular wall 415 of the implant 405 includes an anterior portion 415a, a posterior portion 415b and lateral portions 415c. The implant 405 is made from a non-metal synthetic material with a metal plate 420 integrally formed within the anterior side of the non metal synthetic material implant 405. The metal plate 420 does not fully extend around the implant 405. The non-metal synthetic material may be made from carbon, PEEK (polyethylketone), graphite, woven carbon, Kevlar, or other suitable synthetic material that has strength capable of withstanding compression and rotational forces. The metal material may be titanium, stainless steel or cobaltlchromium. The amalgam feature could also be applied to NMSM threaded cages placed in the anterior lumbar spine, as well as cages placed in the interbody space from a lateral approach. This amalgam feature may also apply to cages, cylindrical or rectangular placed in the cervical or thoracic spine. While the preferred shape of the implant is oval, other shapes may also be used, such as circular, kidney or “D” shaped.

There are numerous advantages of a NMSM/metal amalgam for an implant. For example, one advantage is the metal within the device allows a surgeon to identify the position of the device in space to assist in implantation at the proper location and orientation with in the spine. Another advantage is that the NMSM material allows a surgeon to assess fusion postoperatively after the implantation of the device. This is due to the fact that x-rays penetrate the NMSM to allow bony visualization through the device. The surgeon would be able to evaluate the fusion of the device to the spine by using the lateral x-rays taken only through lateral portions of the NMSM device alone.

One weakness of using a NMSM device alone (i.e., without metal) is the difficulty in holding the device with instruments or less durable antirotation feature. Often the holding instruments (typically made of PEEK) overwhelm the NMSM device during implantation, resulting in deformation and damage. Another advantage of the disclosed NMSM/metal amalgam is that the metal can allow a firmer “grabbing” of the device with implantation tools. Holding or grabbing the proposed NMSM/metal amalgam device with an implantation tool, which would hold the metal plate(s), would avoid such damage to the implant and allow better control during implantation. Advantage of more durable feature to prevent rotation between the implant and plate. The combination of metal in the form of a fixation element within the NMSM device is a novel concept. In some embodiments, a plurality of tool engaging openings (not shown) may be disposed in the annular wall 415 having the metal plate(s) 420. The openings can be threaded or otherwise configured to receive a conventional insertion tool (not shown).

FIGS. 6B-6E show other embodiments of a NMSM/metal amalgam implant. In FIG. 6B, the metal within the device may include one or more metal plates, for example, plates 420a, 420b. In FIG. 6C, the metal within the NMSM/metal amalgam device could take the form of multiple washers or threaded inserts 425. In FIG. 6D, the metal within the NMSM/metal amalgam device could take the form of a plate 430 with threaded screw holes 435. In FIG. 6E, the metal within the NMSM/metal amalgam device could include both anterior plate(s) 420 on the anterior side and posterior plate(s) 440 on the posterior side of the device.

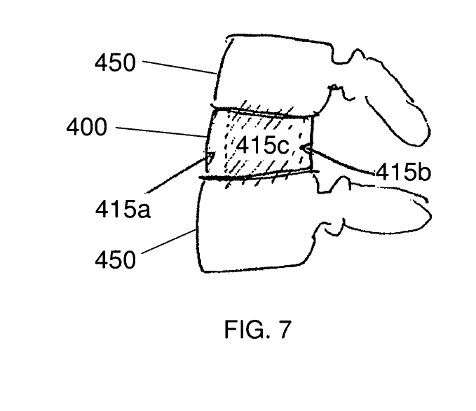

FIG. 7 shows a view of a lateral x-ray showing the implant 400 positioned within between adjacent vertebrae 450. By positioning the metal within the anterior portion 415a, and optionally the posterior portion 415b, the surgeon would be able to evaluate the fusion of the device to the spine by using the lateral x-rays taken only through lateral portions 415c of the NMSM device alone. An anterior 415a/posterior 415b x-ray would not be as desirable as the metal components of the device would obscure the fusion. There are numerous advantages of a NMSM/metal amalgam for an implant. For example, one advantage is the metal within the device allows a surgeon to identify the position of the device in space to assist in implantation at the proper location and orientation with in the spine. Another advantage is that the NMSM material allows a surgeon to assess fusion postoperatively after the implantation of the device. This is due to the fact that x-rays penetrate the NMSM to allow bony visualization through the device. Surgeons typically do not assess fusion through an anterior/posterior x-ray, and this is the view of the fusion that the metal components of the device would obscure.

Example embodiments of the methods and components of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

1. A modular spinal implant system for use between adjacent vertebrae near vascular anatomy, comprising:
an implant configured to fit between adjacent vertebrae, the implant having annular side walls with upper and lower surfaces configured to enclose a hollow interior; and
an attachment plate rotatably coupled to the implant and configured to rotate to variable orientations relative to the implant to avoid the vascular anatomy, the attachment plate having a superior portion that is narrower than an inferior portion, the attachment plate having at least one vertebra attachment hole configured for attaching to at least one adjacent vertebrae using one or more bone screws.

2. The system of claim 1, wherein the attachment plate is selected from a variety of attachment plates configured to avoid the vascular anatomy proximate the vertebrae.

3. The system of claim 1, wherein the inferior portion includes one or more vertebra attachment holes.

4. The system of claim 1, wherein the superior portion includes one vertebra attachment hole and the inferior portion includes two vertebra attachment holes.

5. The system of claim 1, wherein the implant material is selected from the group consisting of titanium, stainless steel, cobalt-chromium, carbon, PEEK (polyethylketone), graphite, woven carbon, Kevlar, and other suitable synthetic material.

6. The system of claim 1, wherein the implant is made of a non metal synthetic material.

7. The system of claim 1, wherein the implant further includes one or more metal plates integrally formed within an anterior portion of the annular side wall.

8. The system of claim 1, wherein the attachment plate is made from titanium, stainless steel, or cobalt-chromium.

9. The system of claim 1, wherein mating surfaces between the attachment plate and implant include an interlock configuration.

10. The system of claim 1, further comprising a bone autograft, allograft or a bone graft substitute positioned within the hollow interior of the implant.

11. A method of installing a modular spinal implant system between adjacent vertebrae, comprising:
inserting the modular spinal implant system between adjacent vertebrae, the system comprising:
an implant configured to fit between adjacent vertebrae, the implant having annular side walls with upper and lower surfaces configured to enclose a hollow interior; and
an attachment plate rotatably coupled to the implant before, during, or after implantation and configured to rotate to variable orientations relative to the implant to avoid the vascular anatomy, the attachment plate having a superior portion that is narrower than an inferior portion, the attachment plate having at least one vertebra attachment hole configured for attaching to at least one adjacent vertebrae using one or more bone screws;
rotating the attachment plate to avoid the vascular anatomy; and
attaching the attachment plate to at least one adjacent vertebra using one or more bone screws.

12. The method of claim 11, wherein the attachment plate is selected from a variety of attachment plates configured to avoid the vascular anatomy proximate the vertebrae.

13. The method of claim 11, wherein prior to inserting the system, the method further includes retracting a portion of the vascular anatomy.

14. The method of claim 11, wherein the implant material is selected from the group consisting of titanium, stainless steel, cobalt-chromium, carbon, PEEK (polyethylketone), graphite, woven carbon, Kevlar, and other suitable synthetic material.

15. The method of claim 11, further comprising filling the hollow interior with bone autograft, allograft or a bone graft substitute.
